(19) United States
(12) Patent Application Publication
(10) Pub. No.: US 2008/0171946 A1
(43) Pub. Date: Jul. 17, 2008

(54) PROCESS AND MEASURING INSTRUMENT FOR DETERMINING THE RESPIRATION RATE

(75) Inventor: Hans-Ullrich HANSMANN, Barnitz (DE)

Correspondence Address: MCGLEW & TUTTLE, PC, P.O. BOX 9227, SCARBOROUGH STATION, SCARBOROUGH, NY, 10510-9227, US

(73) Assignee: DRAGER MEDICAL AG & CO. KG, Lubeck (DE)

(21) Appl. No.: 11/953,341

(22) Filed: Dec. 10, 2007

(30) Foreign Application Priority Data
Jan. 11, 2007 (DE) 10 2007 001 709.1

(51) Int. Cl. A61B 5/053; A61B 5/08

(52) U.S. Cl. 600/529; 600/547

(57) ABSTRACT

A process for determining the respiration rate of a patient by means of vessel plethysmography. Provisions are made according to the present invention for determining the electric impedance between at least two electrodes by means of a control and analysis unit connected to the body via a plurality of electrodes, for which the control and analysis unit is set up to send an alternating voltage through the body and to determine an indicator of the impedance between at least two electrodes, and to automatically record and evaluate the determined value for the indicator of the impedance as a function of the time in order to determine the respiration rate therefrom.

### CROSS REFERENCE TO RELATED APPLICATIONS

This application claims the benefit of priority under 35 U.S.C. §119 of German Patent Application DE 10 2007 001 709.1 filed Jan. 11, 2007, the entire contents of which are incorporated herein by reference.

### FIELD OF THE INVENTION

The present invention pertains to a process and to a measuring instrument for determining the respiration rate by means of vessel plethysmography.

### BACKGROUND OF THE INVENTION

The respiration rate is an important indicator of the health status of a patient. The determination of the respiration rate is carried out with the currently available monitors mostly only in the area of intensive care. Either mechanical respirators or respiration-supporting devices are available for this. Measurements are carried out by means of chest and belly belts or impedance measurements in case of patients without access to the airways by means of masks or tubes.

Processes in which the respiration rate is inferred from the pressure in a vessel by means of photoplethysmography are known from the literature (e.g., from “What is the best site for measuring the effect of ventilation on the pulse oximeter waveform,” Kirk H. Shelly et al., Anesths. Analg., 2006, Vol. 103, pp. 372-377). Vessel plethysmography is used, in general, to determine the dilation of vessels as well as the curve shape, amplitude, frequency and course of this dilation as a function of time. Important information can be obtained from this on the state of the vessels, the cardiovascular system in general and the patient's water balance. The fact that light, preferably light of different wavelengths, is absorbed as a function of the degree of oxygen saturation of hemoglobin and the respiration rate can be directly inferred from the determination of this degree of saturation as a function of the time is utilized in photoplethysmography. This process is very widespread as an SpO₂ measurement and is used in prior-art measuring instruments.

The photoplethysmography process is especially suitable for patients whose oxygen saturation must be monitored for physiological reasons anyway. It must be borne in mind in this connection that the simple determination of the oxygen saturation requires only the short-term measurement of some curves of respiration within a few minutes and hence a short on-time of a total of only a few seconds. By contrast, continuous measurement over a few minutes is necessary for the determination of the respiration rate.

The drawback is that, according to the state of the art, autarchic energy supply is not possible for the continuous operation of photoplethysmography, especially for portable instruments, without considerable restriction of a mobile patient's freedom of movement.

Electroimpedance plethysmography is another special form of vessel plethysmography. The impedance of a body segment is measured here in the surroundings of a vessel, e.g., at the collarbone. The blood in the vessel differs from the surrounding tissue due to a markedly lower impedance, so that the impedance measured in the environment is very strongly affected by the dilation of the vessel. Processes for determining the cardiac activity by observing the dilation of vessels as a function of time are known from the literature (e.g., “Apparative Gef\dot{a}βdiagnostik [Instrumental Diagnostic Procedures on Vessels], Ralf Schüler, ISBN 3-932633-16-4).

### SUMMARY OF THE INVENTION

The object of the present invention is to develop a process and a measuring instrument for the continuous determination of the respiration rate of a mobile patient without appreciably limiting the patient by the measuring process, the analysis or the energy supply for the measuring instrument.

According to the invention, a process is provided for determining the respiration rate of a patient by means of vessel plethysmography. The process comprises the steps of:

setting up a control and analysis unit to apply and send an alternating voltage through the body;

determining an electric impedance between at least two electrodes by means of the control and analysis unit connected to the body via a plurality of electrodes; and

automatically recording and analyzing the determined values for the indicator of the impedance as a function of the time in order to determine the respiration rate therefrom.

Each of the alternating voltage applied and the recording and analyzing of the measured signals may advantageously be carried out in continuous operation. The heart rate may also be determined from the measured signals by means of plethysmography. The measured signals may advantageously be analyzed automatically by means of analog filtration and a fast Fourier transformation method.

The measured signals may be analyzed automatically by means of analog filtration and a digital correlation method. The measured signals may be analyzed automatically by means of analog filtration and a digital filter tuning method. The measured signals may be analyzed automatically by means of analog filtration and a digital lock-in method. The measured signals may be analyzed automatically by means of digital filters.

A value for the reliability of the measurement results may also be provided during the analysis of the measured signals in case an error is determined.

At least two additional measuring electrode pairs may advantageously be arranged, spaced apart from one another by a certain amount, on the body segment through which the current flows, and are connected to the control and analysis unit, and the run time of the pulse wave for the section between the measuring electrode pairs is measured. Two measuring electrode pairs may be arranged at the greatest possible distance between electrodes for feeding the alternating voltage. A value for the reliability of the determination of the pulse wave time may also be provided during the determination of the pulse wave run time based on an error determination.

According to another aspect of the invention, a measuring instrument is provided for determining the respiration rate of a patient by means of vessel plethysmography. The measuring instrument comprises a plurality of electrodes and a control and analysis unit connected to the body via the plurality of electrodes. The control and analysis unit sends an alternating voltage through the body and determines an indicator of the impedance between at least two of the electrodes. The control and analysis unit automatically records and analyzes the determined values as an indicator of the impedance as a function of the time in order to determine the respiration rate therefrom.

The control and analysis unit may carry out continuously both the application of the alternating voltage and the recording and the analysis of the measured signals.

The control and analysis unit may determine the heart rate from the measured signals by means of plethysmography.

The control and analysis unit may process and analyze the measured signals automatically by means of analog filtration and a fast Fourier transformation method. The control and analysis unit may also process and analyze the measured signals automatically by means of analog filtration and a digital correlation method. The control and analysis unit may also process and analyze the measured signals automatically by means of analog filtration and a digital filter tuning method. The control and analysis unit may process and analyze the measured signals automatically by means of analog filtration and a digital lock-in method. The control and analysis unit may also process and analyze the measured signals automatically by means of digital filters.

The control and analysis unit may advantageously provide values for the reliability of the measured results during the recording and the analysis of the measured signals.

At least two additional measuring electrode pairs may be arranged, spaced apart from one another by a certain amount, on the body segment through which the current flows and are connected to the control and analysis unit, and the control and analysis unit measures the run time of the pulse wave for the section between the measuring electrode pairs. The control and analysis unit then may provide a value for the reliability of determination of the pulse wave time during the determination of the pulse wave run time.

The present invention will be explained below on the basis of exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a diagram showing the measured signal and its components on the basis of the heart beat and respiration as a function of the time;

FIG. 2 is a diagram showing an autocorrelation of the measured signal; and

FIG. 3 is view showing of a possible placement of the electrodes on the patient's body.

### DESCRIPTION OF THE PREFERRED EMBODIMENTS

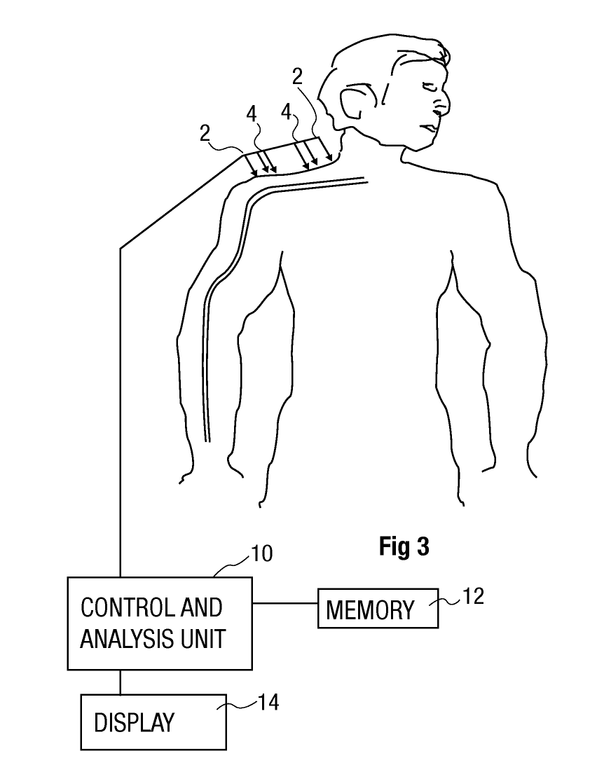

Referring to the drawings in particular, FIG. 3 shows the arrangement of the electrodes 2, 4 in the region of the collarbone of the patient. The electrodes 2, 4 are arranged in a row in the area of a vessel extending laterally along the shoulder. The electrodes 2, 4 are provided for as a 6-electrode array, as it is needed for the determination of the pulse wave run time. The feeding electrodes 2 each are associated with one measuring electrode pair 4 and are arranged in series on the outside of the patient and upstream relative to the direction of blood flow. Another measuring electrode pair is arranged correspondingly downstream relative to the direction of blood flow.

A process and a measuring instrument for determining the respiration rate by means of electroimpedance plethysmography are provided according to the present invention, in which the technical difficulty of determining the respiration rate from the dependence of the pressure in the vessel on the thoracic pressure is overcome by the analysis process described below and nearly wattless, battery-operated operation of a mobile measuring instrument is preferably possible.

The order of magnitude of the thoracic pressure depends on the breathing pattern of the spontaneously breathing patient. Shallow, rapid breathing generates small changes in pressure of about 5 mbar during expiration and −5 mbar during inspiration. Deep, rapid breathing may reach pressure changes of ±20 mbar. Increased airway resistance increases the amplitude of the pressure change in case of the same breathing pattern. The respiration frequencies are between 0.1 Hz and 1 Hz and usually have no harmonic waves, because the stimulations by motions of the chest or diaphragm have a low harmonic content. The pressure changes are superimposed to the markedly greater pressure change that originates from the transportation of blood and alternates with the heart rate. The amplitude of the pressure change of the transportation of blood is 100 mbar to 270 mbar, the frequencies being between 1 Hz and 3 Hz and having marked harmonics as double and triple harmonic frequencies. The vascular system acting as a low-pass filter reduces other higher frequencies. Especially short-term changes in the surrounding tissue, e.g., of the muscles, must be borne in mind as external disturbance variables. The changed external tension affects the dilatability of the vessels and the pressure in the vessel may appear to be reduced, for example, in case of an increase in the external pressure of the muscles, without the pressure conditions in the vessel having, in principle, changed. It is difficult to clearly distinguish the causes from the effect of the thoracic pressure especially in case of external pressure effects that occur periodically at a frequency in the range of the respiration rate during the measurement period. This difficulty can be solved by an analysis of the second and third harmonics, which are associated with the transportation of blood and which often have steeper gradients and hence also larger harmonic components in case of external effects. The signals measured during the measurement of the impedance are automatically processed and analyzed such that this makes it possible to determine the respiration rate.

The measuring instrument for determining the respiration rate by means of electroimpedance plethysmography comprises a control and analysis unit 10. The measurement instrument may also include a memory 12 for recording or storing measured values and output from the processing and analyzing by the control and analysis unit 10 and may include a display 14. The control and analysis unit 10 is connected to the patient's body via at least two electrodes 2, 4 and by which an alternating current is fed, e.g., in the range of a few μA with an output of approx. 0.05 μW; the current is fed such that the current density and the blood flow direction in the body region through which the current flows form one axis. The selected body region is used to measure the vessel dilation and should preferably be subject to little external pressure changes due to muscle motions, as this is the case, e.g., in the area of the collarbone as shown in FIG. 3.

The measurement is preferably carried out via an additional pair of measuring electrodes in a four-electrode array. The advantage of this is a nearly currentless measurement of the voltage drop, which can be determined independently from the contact resistances between the electrodes and the skin. The measured signal consists of an a.c. voltage on a carrier frequency of the alternating current fed, equaling, for example, 50 kHz, as an indicator of the impedance of the body region through which the current flows. The possibility of signal processing by means of filtration, smoothing, offset elimination, etc., is guaranteed due to the fact that the carrier frequency of 50 kHz to 100 kHz is high, contrary to the frequencies of the pressures in the vessels, which latter frequencies are to be determined. The frequency range to be determined, with up to 10 Hz, reaches the third harmonic of the blood transportation. The marked signal of the heart rate is preferably used as a quality indicator for the determination of the alternating voltage. The scanning rate is consequently 100 Hz or higher. The amplitude of the a.c. voltage signal is modulated with the lumen of the vessel, a low amplitude corresponding to a narrow vessel status and dilation of the vessel leading to an increase in signal amplitude. The raw signal is demodulated by folding the signal with the carrier frequency and a useful signal is extracted. Four advantageous embodiments of the process are suitable for the recognition of individual discrete frequencies during the subsequent analysis of the useful signal recorded.

In a first advantageous embodiment of the process, a fast Fourier transformation is used, and the signal is transformed as a function of the time into a function of the frequency. To reduce artifacts, longer signal patterns must be examined. The frequency spectrum of the signal is obtained from the fast Fourier transformation, but the phase position of the signal is not.

In another advantageous embodiment of the process, an autocorrelation is used, where a section of the signal is multiplied by itself and added up, after it was offset by a time. This procedure is repeated for all the times that are of interest for the frequency range and the correlation is plotted as a sum as a function of the time offset. A high sum is obtained with great overlap with the periodicity of the signal.

In another advantageous embodiment of the process, a lock-in process is used, where the useful signal is multiplied by a periodic signal of a fixed frequency and amplitude. The pattern of the amplitude component is obtained for this frequency, and the phase angle can be determined as well. This procedure is repeated with the frequencies of interest and the amplitudes are plotted as a function of the frequency, so that the spectral components of the signal appear.

Tunable filters are used in another advantageous embodiment of the process, and the signal is rated with a band pass filter. This rating is performed in the entire frequency range of interest for individual frequency bands. An amplitude and phase ratio can thus be determined for harmonic frequencies by means of a parallel connection of several filters.

The above-described four advantageous embodiments are suitable for the determination of both the heart rate and the respiration rate.

However, the a.c. voltage signal to be measured can also be additionally analyzed for determining a pulse wave run time. The voltage drop is to be measured for this by means of at least two measuring electrode pairs 4 in addition to the two feeding electrodes 2 in a six-electrode array as shown in FIG. 3. The two measuring electrode pairs are preferably arranged on the axis between the feeding electrodes at a distance of at least a few centimeters on the body. One pair is used to measure the a.c. signals at a point located upstream relative to the blood flow in the vessel and the other pair is used to measure the same signal at a point located downstream relative to the blood flow in the vessel. Due to the run time of the pulse wave in the vessel, the signal is offset by a time offset t from the downstream point to the upstream point. Both signals are multiplied by the carrier signal or filtered in another manner and amplified such that it is standardized to the carrier signal. The adaptation of amplification is carried out slowly at a frequency of less than 0.1 Hz compared to the pulse wave frequencies being considered in the range of 1 Hz. The signal processing is checked by comparing the signals by means of subtraction or division. The pulse wave velocity v is calculated from the time offset t and the locus offset L with v=L/T, where L is obtained from the distance between the measuring electrode pairs and is either estimated, measured or set by a common, fixed connection.

In case of known blood pressure, the pulse wave velocity is an indicator of the elasticity of the vessel or, if unchanged elasticity of the vessel is assumed, an indicator of the blood pressure. An absolute indicator of the blood pressure can be determined continuously on the basis of this indicator for the blood pressure after calibration with an independent blood pressure measurement, e.g., by an oscillatory blood pressure measurement by means of an upper arm cuff. The advantage is the considerable reduction of the compromise for the patient, especially for his freedom of motion.

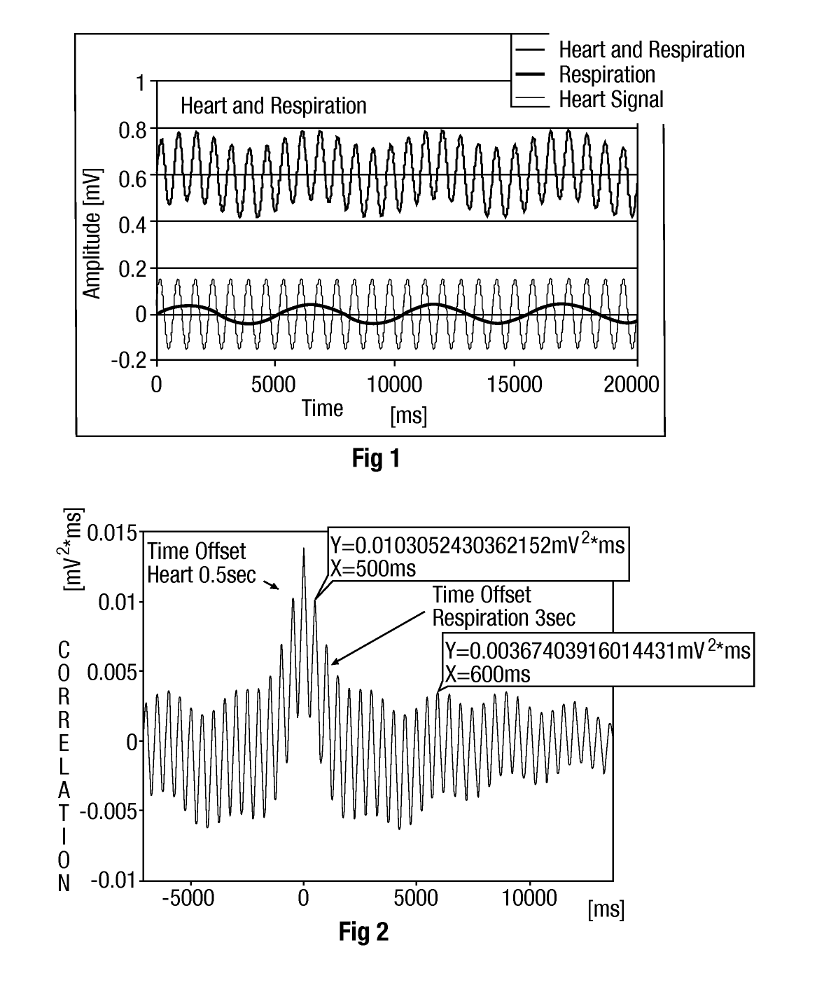

The upper curve in FIG. 1 shows the measured value, a recording of the a.c. voltage signal in m V as a function of the time in milliseconds (msec) after folding the raw signal with the carrier frequency, i.e., after the carrier frequency signal components have been removed. This measured or useful signal, obtained from the folding with the carrier frequency, is composed of the main frequency components for the (heart) cardiac activity (1-3 Hz) and for respiration (0.1-1 Hz). These frequency components of the measured signal are shown in the lower curves and are designated as “heart signal” and “respiration” in the legend.

FIG. 2 shows the result of the autocorrelation of the useful signal in units of mV²·msec as a function of the time offset τ in msec used for the autocorrelation. Noise and interference signals converge in the area of low values for τ, so that a signal-to-noise ratio can be read there. A respiration rate of 0.33 Hz is seen as a periodical amplitude modulation of the correlation for high values of τ after elimination of interfering frequencies. The superimposed modulation with a period duration of τ=0.5 sec corresponds to a heart rate of 2 Hz.

While specific embodiments of the invention have been shown and described in detail to illustrate the application of the principles of the invention, it will be understood that the invention may be embodied otherwise without departing from such principles.

What is claimed is:

1. A process for determining the respiration rate of a patient by means of vessel plethysmography, the process comprising the steps of:
setting up a control and analysis unit to apply and send an alternating voltage through the body;
determining an electric impedance between at least two electrodes by means of the control and analysis unit connected to the body via a plurality of electrodes; and
automatically recording and analyzing the determined values for an indicator of the impedance as a function of the time in order to determine the respiration rate therefrom.

2. A process in accordance with claim 1, wherein both the alternating voltage applied and the recording and analyzing of the measured signals are carried out in continuous operation.

3. A process in accordance claim 1, wherein a heart rate is also determined from the measured signals by means of plethysmography.

4. A process in accordance claim 1, wherein the measured signals are analyzed automatically by means of analog filtration and a fast Fourier transformation method.

5. A process in accordance with claim 1, wherein the measured signals are analyzed automatically by means of analog filtration and a digital correlation method.

6. A process in accordance with claim 1, wherein the measured signals are analyzed automatically by means of analog filtration and a digital filter tuning method.

7. A process in accordance with claim 1, wherein the measured signals are analyzed automatically by means of analog filtration and a digital lock-in method.

8. A process in accordance with claim 1, wherein the measured signals are analyzed automatically by means of digital filters.

9. A process in accordance claim 1, wherein a value for the reliability of the measurement results is also provided during the analysis of the measured signals in case an error is determined.

10. A process in accordance claim 1, wherein at least two additional measuring electrode pairs are arranged, spaced apart from one another by a certain amount, on the body segment through which the current flows, and are connected to the control and analysis unit, and the run time of the pulse wave for the section between the measuring electrode pairs is measured.

11. A process in accordance with claim 10, wherein two measuring electrode pairs are arranged at a greatest possible distance between electrodes for feeding the alternating voltage.

12. A process in accordance with claim 10, wherein a value for the reliability of the determination of the pulse wave time is also provided during the determination of the pulse wave run time based on an error determination.

13. A measuring instrument for determining the respiration rate of a patient by means of vessel plethysmography, the measuring instrument comprising:
a plurality of electrodes;
a control and analysis unit connected to the body of the patient via said plurality of said electrodes, said control and analysis unit sending an alternating voltage through the body and determining an indicator of the impedance between at least two of said electrodes, and automatically recording and analyzing the determined values as the indicator of the impedance as a function of the time in order to determine the respiration rate therefrom.

14. A measuring instrument in accordance with claim 13, wherein the control and analysis unit is set up to carry out continuously both the application of the alternating voltage and the recording and the analysis of the measured signals.

15. A measuring instrument in accordance with claim 13, wherein the control and analysis unit determines the heart rate from the measured signals by means of plethysmography.

16. A measuring instrument in accordance with claim 13, wherein the control and analysis unit processes and analyzes the measured signals automatically by means of analog filtration and a fast Fourier transformation method.

17. A measuring instrument in accordance with claim 13, wherein the control and analysis unit processes and analyzes the measured signals automatically by means of analog filtration and a digital correlation method.

18. A measuring instrument in accordance with claim 13, wherein the control and analysis unit processes and analyzes the measured signals automatically by means of analog filtration and a digital filter tuning method.

19. A measuring instrument in accordance with claim 13, wherein the control and analysis unit processes and analyzes the measured signals automatically by means of analog filtration and a digital lock-in method.

20. A measuring instrument in accordance with claim 13, wherein the control and analysis unit processes and analyzes the measured signals automatically by means of digital filters.

21. A measuring instrument in accordance with claim 13, wherein the control and analysis unit provides values for the reliability of the measured results during the recording and the analysis of the measured signals.

22. A measuring instrument in accordance with claim 13, wherein at least two additional measuring electrode pairs are arranged, spaced apart from one another by a certain amount, on the body segment through which the current flows and are connected to the control and analysis unit, and the control and analysis unit measures the run time of the pulse wave for the section between the measuring electrode pairs.

23. A measuring instrument in accordance with claim 22, wherein the control and analysis unit provides a value for the reliability of determination of the pulse wave time during the determination of the pulse wave run time.
